(19) United States
(12) Patent Application Publication
(10) Pub. No.: US 2003/0235576 A1
(43) Pub. Date: Dec. 25, 2003

(54) New drug combinations for the treatment of ischaemic conditions

(75) Inventors: Hermann Duettmann, Gau-Algesheim (DE); Annerose Mauz, Langenenslingen-Emerfeld (DE); Sophie Banzet, Mennisweiler-Bad Waldsee (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/460,709

(22) Filed: Jun. 12, 2003

Related U.S. Application Data
(60) Provisional application No. 60/408,144, filed on Sep. 4, 2002.

(30) Foreign Application Priority Data
Jun. 15, 2002 (DE) 10226814

(51) Int. Cl. A61K 38/48; A61K 31/473

(52) U.S. Cl. 424/94.64; 514/295

(57) ABSTRACT

The invention relates to new drug combinations based on sodium channel blockers 1 and fibrinolytics 2, processes for the preparation thereof as well as the use thereof for preparing pharmaceutical compositions for the treatment of ischaemic conditions.

The invention relates to new drug combinations based on sodium channel blockers 1 and fibrinolytics 2, processes for the preparation thereof as well as the use thereof for preparing pharmaceutical compositions for the treatment of ischaemic conditions.

### DESCRIPTION OF THE INVENTION

The invention relates to drug combinations containing one or more, preferably one sodium channel blocker 1 and one or more, preferably one fibrinolytic 2, optionally in the presence of conventional excipients or carriers.

A) Sodium channel blockers 1 which may be used according to the invention:

Within the scope of the present invention preferred sodium channel blockers 1 are those selected from the group consisting of pirmencol, sipatrigine, irampanel, pilsicainide, oxcarbazepine, topiramate, fosphenytoin, flunarizin, ropivacaine, levobupivacaine, zonisamide, mexiletine, bipridil, bisaramil, milacainide, safinamide, bupivacaine, tetrodotoxin, NS 7, the compounds of general formula 1a

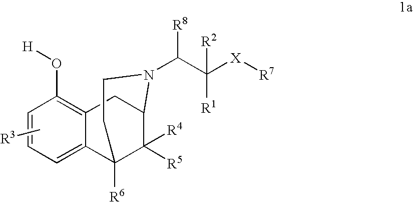

wherein

X denotes a single bond, —O, C₁-C₄-alkylene, an alkylene bridge with 1 to 8 carbon atoms which may be branched or unbranched and may have at any point in the bridge one or two oxygen atom(s) or a nitrogen atom, preferably O—C₁-C₃-alkylene or —O-CH₂-CH₂—O, —O—CH₂—CH₂—NH—;

R¹ denotes hydrogen, methyl, ethyl, phenyl;

R² denotes hydrogen, methyl;

R³ denotes hydrogen, fluorine, chlorine, bromine, hydroxy, methyl, methoxy;

R⁴ denotes hydrogen, methyl, ethyl;

R⁵ denotes hydrogen, methyl, ethyl;

R⁶ denotes hydrogen, methyl, ethyl;

R⁷ denotes tert.-butyl, cyclohexyl or phenyl, while phenyl may optionally be substituted by R⁹ and R¹⁰, which may be identical or different;

R⁸ denotes hydrogen, C₁-C₄-alkyl;

R⁹ denotes hydrogen, methyl, fluorine, chlorine, bromine, methoxy;

R¹⁰ denotes hydrogen, methyl, fluorine, chlorine, bromine, methoxy;

optionally in the form of the individual optical isomers, mixtures of the individual enantiomers or racemates as well as in the form of the free bases or the corresponding acid addition salts with pharmacologically acceptable acids;

and the compounds of general formula 1b.

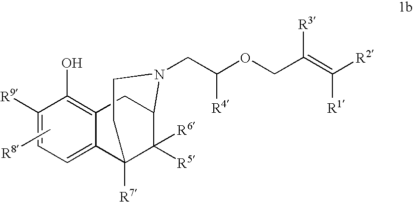

wherein

R^{1′}, R^{2′} and R^{3′} which may be identical or different, denote hydrogen, methyl or ethyl;

R^{4′} denotes hydrogen, methyl or ethyl;

R^{5′}, R^{6′} and R^{7′} which may be identical or different, denote hydrogen, methyl or ethyl;

R^{8′} and R^{9′} which may be identical or different, denote hydrogen, fluorine, chlorine, bromine, methyl, ethyl, hydroxy or methoxy,

optionally in the form of the racemates, the enantiomers, the diastereomers and the mixtures thereof, and optionally the pharmacologically acceptable acid addition salts thereof.

Particularly preferred within the scope of the present invention is or are the sodium channel blocker(s) 1 selected from the group consisting of pirmencol, pilsicainide, sipatrigine, irampanel, fosphenytoin, zonisamide, mexiletine, bipridil, bisaramil, milacainide, NS 7, the compounds of general formula 1a wherein

X denotes C₁-C₃-alkylene, —O—CH₂—CH₂—O or-—O—CH₂—CH₂—NH—;

R¹ denotes hydrogen or methyl;

R² denotes hydrogen or methyl;

R³ denotes hydrogen or chlorine;

R⁴ denotes hydrogen or methyl;

R⁵ denotes hydrogen or methyl;

R⁶ denotes methyl or ethyl;

R⁷ denotes tert.-butyl, cyclohexyl or phenyl, while phenyl may optionally be substituted by R⁹ and R¹⁰, which may be identical or different;

R⁸ denotes hydrogen;

R⁹ denotes hydrogen, methyl, fluorine or chlorine;

R¹⁰ denotes hydrogen, methyl, fluorine or chlorine;

optionally in the form of the individual optical isomers, mixtures of the individual enantiomers or racemates as well as in the form of the free bases or the corresponding acid addition salts with pharmacologically acceptable acids;

and the compounds of general formula 1b, wherein

R^{1′}, R^{2′} and R^{3′} which may be identical or different, denote hydrogen or methyl;

R^{4′} denotes hydrogen or methyl;

R^{5′}, R^{6′} and R^{7′} which may be identical or different, denote hydrogen or methyl, preferably methyl;

R^{8′} denotes hydrogen, methyl, hydroxy or methoxy, preferably hydrogen or methyl,

R^{9′} denotes hydrogen or methyl,

optionally in the form of the racemates, the enantiomers, the diastereomers and the mixtures thereof, and optionally the pharmacologically acceptable acid addition salts thereof.

Particularly preferred within the scope of the present invention is or are the sodium channel blocker(s) 1 selected from the group consisting of fosphenytoin, zonisamide, sipatrigine, irampanel, mexiletine, NS 7, the compounds of general formula 1a wherein

X denotes C₁-C₃-alkylene or —O—CH₂—CH₂—O—;

R¹ denotes hydrogen or methyl;

R² denotes hydrogen or methyl;

R³ denotes hydrogen;

R⁴ denotes hydrogen or methyl;

R⁵ denotes hydrogen or methyl;

R⁶ denotes methyl;

R⁷ denotes phenyl, the phenyl may optionally be substituted by R⁹ and R¹⁰, which may be identical or different;

R⁸ denotes hydrogen;

R⁹ denotes hydrogen, methyl, fluorine or chlorine;

R¹⁰ denotes hydrogen, methyl, fluorine or chlorine;

optionally in the form of the individual optical isomers, mixtures of the individual enantiomers or racemates as well as in the form of the free bases or the corresponding acid addition salts with pharmacologically acceptable acids;

and the compounds of general formula 1b wherein

R^{1′}, R^{2′} and R^{3′} which may be identical or different, denote hydrogen or methyl;

R^{4′} denotes hydrogen or methyl;

R^{5′}, R^{6′} and R^{7′} denote methyl;

R^{8′} denotes hydrogen or methyl, preferably hydrogen;

R^{9′} denotes hydrogen or methyl,

optionally in the form of the racemates, the enantiomers, the diastereomers and the mixtures thereof, and optionally the pharmacologically acceptable acid addition salts thereof.

C₁-C₄-alkyl or C₁-C₈-alkyl generally denotes a branched or unbranched hydrocarbon group with 1 to 4 or 1 to 8 carbon atom(s), which may optionally be substituted by one or more halogen atoms—preferably fluorine—which may be identical to or different from one another. The following hydrocarbon groups are mentioned by way of example: methyl, ethyl, propyl, 1-methylethyl (isopropyl), n-butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2,-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Unless otherwise stated, lower alkyl groups with 1 to 4 carbon atoms, such as methyl, ethyl, propyl, isopropyl, n-butyl, 1-methylpropyl, 2-methylpropyl or 1,1-dimethylethyl, are preferred.

Accordingly, alkylene denotes a branched or unbranched double-bonded hydrocarbon bridge with 1 to 8 carbon atoms, which may optionally be substituted by one or more halogen atoms—preferably fluorine—which may be identical to or different from one another.

Alkoxy generally denotes a straight-chain or branched hydrocarbon group bonded via an oxygen atom—a lower alkoxy group with 1 to 4 carbon atom(s) is preferred. The methoxy group is particularly preferred.

A particularly preferred compound of formula 1a is (−)-(1R,2″S)-2-(2″-benzyloxy)propyl-4′-hydroxy-5,9,9-trimethyl-6,7-benzomorphan in the form of the free bases or the corresponding acid addition salts with pharmacologically acceptable acids. This compound is also known by the name crobenetine.

Of particular interest are the following compounds of general formula 1b: (2R)-N-allyloxyethyl-1,2,3,4,5,6-hexahydro-6,11,11-trimethyl-2,6-methano-3-benzazocin-10-ol-hydrochloride and (2R,2″S)-N-(2-allyloxy-propyl)-1,2,3,4,5,6-hexahydro-6,11,11-trimethyl-2,6-methano-3-benzazocin-10-ol-hydrochloride.

Accordingly, within the scope of the present invention, the component 1 is most preferably selected from the group consisting of fosphenytoin, zonisamide, sipatrigine, irampanel, mexiletine, NS 7, crobenetine, (2R)-N-allyloxyethyl-1 ,2,3,4,5,6-hexahydro-6,11,11-trimethyl-2,6-methano-3-benzazocin-10-ol-hydrochloride and (2R,2″S)-N-(2-allyloxy-propyl)-1,2,3,4,5,6-hexahydro-6,11,11-trimethyl-2,6-methano-3-benzazocin-10-ol-hydrochloride, most preferably crobenetine, (2R)-N-allyloxyethyl-1,2,3,4,5,6-hexahydro-6,11,11-trimethyl-2,6-methano-3-benzazocin-10-ol-hydrochloride and (2R,2″S)-N-(2-allyloxy-propyl)-1,2,3,4,5,6-hexahydro-6,11,11-trimethyl-2,6-methano-3-benzazocin-10-ol-hydrochloride, while crobenetine is of exceptional importance.

The compounds 1 may optionally be used in the form of their salts, and, particularly for pharmaceutical use, in the form of the pharmacologically acceptable acid addition salts with an inorganic or organic acid. Suitable acids for this include for example succinic acid, hydrobromic acid, acetic acid, fumaric acid, maleic acid, methanesulphonic acid, lactic acid, phosphoric acid, hydrochloric acid, sulphuric acid, tartaric acid or citric acid. It is also possible to use mixtures of the above acids.

The compounds of formula 1a are known from WO 99/14199. The compounds of formula 1b are not yet known in the prior art.

The compounds of formula 1b which may be used according to the invention and are not yet known in the prior art may be prepared analogously to methods of synthesis known per se. Possible methods of synthesising the compounds of formula 1b are described by way of example hereinafter.

### SYNTHESIS EXAMPLE 1

### (2R)-N-allyloxyethyl-1,2,3,4,5,6-hexahydro-6,11,11-trimethyl-2,6-methano-3-benzazocin-10-ol-hydrochloride

1.8 g of allyloxyacetic acid are placed in 15 ml dichloromethane, combined with 4.8 g of TBTU and 7.5 ml of ethyldiisopropylamine and stirred at RT for 15 min. Then the mixture is cooled to −5° C. and 3.1 g of 1,2,3,4,5,6-hexahydro-6,11,11-trimethyl-2,6-methano-3-benzazocin-10-ol are added. The mixture is stirred for 30 min at 0° C., and 1 h at RT. Then it is washed once with 100 ml of 2N HCL and once with 100 ml 10% potassium carbonate solution, dried and evaporated down in vacuo. The residue is taken up in 50 ml of THF and added dropwise under nitrogen to a suspension of 1.0 g of lithium aluminium hydride in 50 ml THF. (Temp. rises to 35° C.). It is then heated to 50° C., stirred for 1 h, cooled and at 0-10° C. 1 ml of water is added dropwise, the mixture is stirred for 30 min, 3 ml of NaOH are added and the mixture is stirred for 30 min. The precipitate is suction filtered, the mother liquor evaporated down in vacuo and the residue is filtered through a short column (approx. 75 ml silica gel; dichloromethane 70, ethyl acetate 20, methanol 10). The appropriate fractions are evaporated down in vacuo and crystallised from acetone+eth.HCl.

Yield 2.8 g of (77%), melting point: 236° C.; [α]_{D}²⁰=−78.3° (c=1; methanol).

### SYNTHESIS EXAMPLE 2

### (2R,2″S)-N-(2-allyloxy-propyl)-1,2,3,4,5,6-hexahydro-6,11,11-trimethyl-2,6-methano-3-benzazocin-10-ol-hydrochloride

This is prepared analogously to the method according to Example 1.

Yield 56%, melting point: 239° C.; [α]_{D}²⁰=−33.9° (c=1; methanol).

### SYNTHESIS EXAMPLE 3

### (2R,2″S)-N-(2-but-2-enoxy-propyl)-1,2,3,4.5,6-hexahydro-6,11,11-trimethyl-2,6-methano-3-benzazocin-10-ol-hydrochloride

This is prepared analogously to the method according to Example 1.

Yield 47%, melting point: 205° C.

### SYNTHESIS EXAMPLE 4

### (2R,2″S)-N-[2-(2-methyl-propenoxy)-propyl]-1,2,3,4,5,6-hexahydro-6,11,11-trimethyl-2,6-methano-3-benzazocin-10-ol-hydrochloride

This is prepared analogously to the method according to Example 1.

Yield 12%, melting point: 240° C.; [α]_{D}²⁰=−29.6° (c=1; methanol).

### SYNTHESIS EXAMPLE 5

### (2R)-N-[2-allyloxy-propyl]-1,2,3,4,5,6-hexahydro-6,11,11-trimethyl-2,6-methano-3-benzazocin-10-ol-hydrochloride

1.9 g of (2R)-N-allyloxyethyl-1,2,3,4,5,6-hexahydro-6,11,11-trimethyl-2,6-methano-3-benzazocin-10-ol-hydrochloride (Example 1) are dissolved in 40 ml of methanol and combined with 3 g of 30% formalin solution and 3 ml of 4 N NaOH. The mixture is heated to 50° C. for 12 hours, the solvent is removed in vacuo, 100 ml of water are added to the residue and it is extracted twice with 200 ml of ether. The organic phase is washed with water, dried and evaporated down in vacuo. The residue is dissolved in 20 ml of dichloromethane and at RT 1.5 g of SOCl₂ added dropwise. After 30 min. it is evaporated down in vacuo, the residue is taken up in 20 ml THF and under nitrogen added dropwise to a suspension of 0.5 g of lithium aluminium hydride in 20ml of tetrahydrofuran. Then it is heated to 50° C. for 2 h, cooled, 1.5 ml of 4N NaOH are added dropwise and the mixture is stirred for 30 min. The precipitate is suction filtered and the mother liquor is evaporated down in vacuo. The residue is filtered through a short silica gel column (approx 30 ml of silica gel, approx 250 ml of ethyl acetate).). The appropriate fractions are evaporated down in vacuo and crystallised from acetone+eth.HCl.

Yield 1.1 g of (56%), melting point: 212° C., [α]_{D}²⁰=−71.6° (c=1; methanol).

### SYNTHESIS EXAMPLE 6

### (2R,2″S)-N-[2-allyloxy-propyl]-1,2,3,4,5,6-hexahydro-6,9,11,11-tetramethyl-2,6-methano-3-benzazocin-10-ol-hydrochloride

This is prepared analogously to the method according to Example 5 starting from Example 2. Yield 60%, melting point: 215° C.; [α]_{D}²⁰=−29.3° (c=1; methanol).

B) Fibrinolytics 2 which maV be used according to the invention:

Within the scope of the present invention the fibrinolytics 2 selected from among the plasminogen activators are preferred. Of particular interest are alteplase (human tissue plasminogen activator, t-PA), tenecteplase, reteplase, streptokinase, urokinase, anistreplase, monteplase, nateplase, duteplase, lanoteplase, silteplase, amediplase and desmoteplase. All these fibrinolytics are known in the art.

Alteplase (amino acid sequence: GenBank Accession No. AAB59510) is a fibrinolytic licensed for use as a drug, the preparation of which by recombinant expression, preferably in cell lines of the Chinese hamster Cricetulus griseus (CHO cells), as well as its pharmaceutical formulation and medicinal use have been described in detail in the prior art (Pennica et al., Nature 301, 214-221 (1983); EP 0 093 619; Andersen et al., Biotechnol Bioeng 70 (1), 25-31 (2000); Dowd et al., Biotechnol Prog 16 (5), 786-794 (2000); Fann et al., Biotechnol Bioeng 69 (2), 204-212 (2000); Werner et al., Arzneimittelforschung 48 (8), 870-880 (1998); Wernicke et Will, Anal Biochem 203 (1),146-150 (1992); Bos et al., Biochim Biophys Acta 1117 (2),188-192 (1992); Dodd et al., FEBS Lett 209 (1), 13-17 (1986); Matsuo et al., J Chromatogr 369 (2), 391-397 (1986); Einarsson et al., Biochim Biophys Acta 830 (1), 1-10 (1985); Kruithof et al., Biochem J 226 (3), 631-636 (1985); Nguyen et al., Pharm Biotechnol. 5, 91-134 (1993); The Gusto III Investigators, N Engl J Med. 1997 October 16;337(16):1118-23; EP 0 239292; WO 86/05514).

Tenecteplase (TN K-tPA; T103N, N117Q, KHRR(296-299)AAAA-tPA) is also a fibrinolytic licensed for use as a drug. Its preparation and use is described in detail in the references WO 93/24635; Keyt et al., Proc Natl Acad Sci USA. 1994 April 26;91 (9):3670-4; Turcasso et Nappi, Ann Pharmacother. 2001 October; 35(10):1233-40; MacGahan, Issues Emerg Health Technol. 2001 January; (13):1-6; Davydov et Cheng, Clin Ther. 2001 July; 23(7):982-97; The Assent II investigators, Lancet. 1999 August 28;354(9180):716-22.

The preparation of reteplase, which is also licensed for drug use, and its pharmaceutical formulations and uses are described in the literature in WO 90/03497; WO 91/08765, WO 91/08766, and Noble et McTavish, Drugs. 1996 October; 52(4):589-605. The preparation, pharmaceutical formulation and medical use of lanoteplase are described in the literature in WO 87/04722 and WO 90/08557. The preparation, pharmaceutical formulation and medical use of desmoteplase are described in the literature in WO 90/09438 and WO 97/29188. The preparation, pharmaceutical formulation and medical use of anistreplase, which is licensed for drug use, is described in the literature in EP 0 028 489, Fears, Semin Thromb Hemost 15 (2),129-139 (1989); Anderson et al., Circulation. 1991 January; 83(1):12640; Been et al., Int J Cardiol. 1986 April; 11(1):53-61; Marder et al., Ann Intern Med. 1986 March; 104(3):304-10; Walker et al, Thromb Haemost. 1984 April 30;51 (2):204-6, and Matsuo et al, Thromb Res Suppl. 1981 November 15;24(4):347-58. Urokinase is described in EP 0 143 949, EP 0 154 272, EP 0 303 028, and EP 0 620 279.

A large number of other plasminogen activators which may be used as component 2 for the combination according to the invention are described in the literature.

Particularly preferred within the scope of the present invention are the fibrinolytics 2 selected from the group consisting of alteplase (t-PA), tenecteplase, reteplase, streptokinase, urokinase, anistreplase, monteplase and nateplase. Particularly preferred fibrinolytics 2 according to the invention are selected from among alteplase (t-PA), tenecteplase, reteplase, urokinase and anistreplase, while alteplase, tenecteplase and reteplase and most preferably alteplase are of exceptional importance according to the invention.

C) Use of the drug combinations of 1 and 2 according to the invention:

The present invention further relates to the use of the combinations according to the invention of one or more, preferably one sodium channel blocker 1 and one or more, preferably one fibrinolytic 2 for preparing a pharmaceutical composition for the treatment of ischaemic conditions of various origins. Preferably, the present invention relates to the use of the combinations according to the invention of one or more, preferably one sodium channel blocker 1 and one or more, preferably one fibrinolytic 2 for preparing a pharmaceutical composition for the treatment of cardiac or cerebral ischaemias, most preferably for the treatment of stroke. Of particular importance within the scope of the present invention is the use of the combinations according to the invention of one or more, preferably one sodium channel blocker 1 and one or more, preferably one fibrinolytic 2 for the treatment of ischaemic stroke, most preferably acute ischaemic stroke.

The present invention further relates to a process for treating ischaemic conditions of various origins which is characterised in that a combination according to the invention of one or more, preferably one sodium channel blocker 1 and one or more, preferably one fibrinolytic 2 is administered. The present invention preferably relates to a method of treating cardiac or cerebral ischaemias, most preferably stroke, and more preferably according to the invention ischaemic stroke, most preferably acute ischaemic stroke, which is characterised in that a combination according to the invention of one or more, preferably one sodium channel blocker 1 and one or more, preferably one fibrinolytic 2 is administered.

The present invention further relates to the use of one or more, preferably one sodium channel blocker 1 for preparing a pharmaceutical composition for the combined treatment of ischaemic conditions of various origins with one or more, preferably one fibrinolytic 2. The present invention preferably relates to the abovementioned use for preparing a pharmaceutical composition for the combined treatment of cardiac or cerebral ischaemias, most preferably for the treatment of stroke with one or more, preferably one fibrinolytic 2. Of particular importance within the scope of the present invention is the present use for the combined treatment of ischaemic stroke, most preferably acute ischaemic stroke with one or more, preferably one fibrinolytic 2.

The present invention further relates to a method of treating ischaemic conditions of various origins which is characterised in that one or more, preferably one sodium channel blocker 1 and one or more, preferably one fibrinolytic 2 are administered simultaneously or sequentially in one single or two separate, preferably in two separate preparations. The present invention preferably relates to a method of treating cardiac or cerebral ischaemias, most preferably stroke, and more preferably according to the invention ischaemic stroke, most preferably acute ischaemic stroke, which is characterised in that one or more, preferably one sodium channel blocker 1 and one or more, preferably one fibrinolytic 2 are administered simultaneously or sequentially in one single or two separate, preferably in two separate preparations.

D.1) Administration of the drug combinations of 1 and 2 according to the invention:

The drug combinations according to the invention may contain the active ingredients 1 and 2 in one single or two separate preparations. In the combinations of zonisamide, mexiletine, NS 7, crobenetine, (2R)-N-allyloxyethyl-1,2,3,4,5,6-hexahydro-6,11,11-trimethyl-2,6-methano-3-benzazocin-10-ol-hydrochloride or (2R,2″S)-N-(2-allyloxy-propyl)-1,2,3,4,5,6-hexahydro-6,11,11-trimethyl-2,6-methano-3-benzazocin-10-ol-hydrochloride as component I with alteplase (t-PA), tenecteplase, reteplase, urokinase or anistreplase as component 2 which are of particular importance according to the invention, the two components are preferably contained in two separate preparations, for example in the form of a so-called kit. Separate formulations of the two components 1 and 2 are described in detail in the following paragraphs.

The combination of 1 and 2 according to the invention may be administered, within the scope of the abovementioned use and within the scope of the abovementioned process by simultaneously administering the combination of 1 and 2 or, when 1 and 2 are present in different preparations, by administering components 1 and 2 simultaneously or sequentially. The term sequentially within the scope of the present invention refers to any method of administering components 1 or 2 which does not take place simultaneously. By simultaneous administration is meant the method of administration in which at least one of components 1 and 2 is administered for example by infusion over a longer period and the other component is also used during this period. If the two components 1 and 2 are both administered by infusion over a longer period of time, the word simultaneously for the purposes of the present invention means that the infusion periods overlap for at least a short time.

Particularly when treating ischaemic stroke, which is the preferred indication within the scope of the present invention, most preferably when treating acute ischaemic stroke, components 1 and 2 are preferably given simultaneously or at least within a short time of each other, i.e. for example within one hour. Treatment with the drug combinations according to the invention is particularly effective when it is given as quickly as possible after the stroke takes place. Preferably, the treatment starts at the latest within about 5 hours, most preferably within 4 hours, more preferably still within 3 hours after the stroke occurs.

D.2) Pharmaceutical formulation and administration of component 1:

Within the scope of the present invention the compounds 1 may be administered orally, transdermally, by inhalation or parenterally. The compounds 1 occur as active ingredients in conventional preparations, for example in compositions which consist essentially of an inert pharmaceutical carrier and an effective dose of the active substance, such as for example tablets, coated tablets, capsules, lozenges, powders, solutions, suspensions, emulsions, syrups, suppositories, transdermal systems etc. An effective dose of the compounds 1, particularly the compounds of formulae 1a and 1b, is between 1 and 1000, preferably between 1 and 500, most preferably between 5-300 mg/dose for oral administration, and between 0.001 and 100, preferably between 0.1 and 70 mg/dose for intravenous or intramuscular administration.

It is particularly possible to use the component 1 according to the invention as a solution for infusion, preferably in a physiological saline or nutrient saline solution. In an infusion, for example 10-100 mg/h, preferably 25-60 mg/h of the compound 1 may be used. This latter method of administration is exceptionally important according to the invention.

The compound 1 crobenetine, which is particularly preferred according to the invention, is most preferably administered intravenously by infusion. Preferably between 10 and 60 mg (based on the free base crobenetine) are administered per dose. It is also possible to administer crobenetine as component 1 of the drug combination according to the invention at different time intervals in different doses for each therapy. For example, crobenetine may be given as component 1 first of all in a dosage of 20-70 mg, preferably 30 to 60 mg, most preferably 50 mg over a period of about 30 minutes to 2 hours, preferably over 45 to 90 minutes, most preferably over one hour. This first administration of component 1 crobenetine may then be followed by further administrations in doses of for example 5 to 50 mg, preferably 10 to 40 mg, most preferably 20 to 30 mg over a period of 2 to 8 hours, preferably 3 to 7 hours, most preferably 4 to 6 hours. If desired this second administration of component 1 crobenetine may be followed others.

Some particular embodiments of formulations for parenteral administration are described hereinafter which may be used in particular for the compounds of formulae 1a and 1b which are particularly preferably used as sodium channel blockers 1.

These formulations contain at least one compound of formula 1a or 1b or one of the pharmaceutically acceptable salts thereof and a cyclodextrin derivative, particularly gamma-cyclodextrin (γ-CD), hydroxypropyl-gamma-cyclodextrin (HP-γ-CD), hydroxypropyl-beta-cyclodextrin (HP-β-CD) or sulphobutylether-beta-cyclodextrin (SBE-μ-CD). The preferred cyclodextrin derivative is hydroxypropyl-γ-cyclodextrin. Hydroxypropyl-γ-cyclodextrin with a molar substitution level of 0.5 to 0.7 is sold for example by Messrs Wacker-Chemie GmbH, D-Burghausen, under the name “CAVASOL® W8 HP Pharma”. “CAVASOL® W8 HP Pharma” is particularly preferred for these pharmaceutical compositions.

Apart from the compound of formula 1a or 1b and the cyclodextrin derivative the pharmaceutical compositions intended for parenteral use according to the invention may contain hydroxy acids such as for example malic acid, lactic acid, tartaric acid or citric acid. They may also optionally contain conventional excipients and carriers such as for example the isotonic agents glucose, mannitol or sodium chloride or sodium acetate or sodium acetate trihydrate as buffer combined with acetic acid or a citric acid/phosphate buffer consisting of e.g. citric acid and disodium hydrogen phosphate or disodium hydrogen phosphate dihydrate. The solvent used is normally water for injections.

The molar ratio of the compound of formula 1a or 1b to cyclodextrin is between 1:1 and 1:5 for example in these formulations. A molar ratio of 1:2.5 to 1:3.5 is preferred. In the presence of hydroxy acid this molar ratio is preferably between 1:0.5 and 1:3 according to the invention; a molar ratio of 1:0.5 to 1:1.5 is particularly preferred. These formulations are most preferably used with crobenetine as component 1.

Apart from the formulations described above containing at least one compound of formula 1a or 1b or one of the pharmaceutically acceptable salts thereof and a cyclodextrin derivative, equally preferred formulations for parenteral use according to the invention are those which contain mannitol as excipient, in addition to a compound of formula 1a or 1b or one of the pharmaceutically acceptable salts thereof. The amount of mannitol is preferably chosen so as to obtain an isotonic solution. These pharmaceutical compositions may optionally also contain other conventional excipients and carriers such as for example an acetic acid/acetate buffer consisting of acetic acid and sodium acetate or sodium acetate-trihydrate or a citric acid/phosphate buffer consisting for example of citric acid and disodium hydrogen phosphate or disodium hydrogen phosphate dihydrate. Usually, the quantity of the buffer components is selected so as to obtain a particular pH value and a particular buffer capacity. The solvent used is normally water for injections.

Preferably, the pharmaceutical composition contains an acetic acid/acetate buffer in addition to the isotonic agent mannitol. A 0.005 to 0.05 molar, preferably a 0.005 to 0.02 molar acetic acid/acetate buffer with a pH of 3.8 to 5 is particularly preferred while a 0.01 molar acetic acid/acetate buffer with a pH value of about 4 is most particularly preferred. The concentration specified relates to the total concentration of acetic acid and acetate; the ratio of acetic acid to acetate results from the desired pH. The pH specified is measured both in the pure buffer solution and in the finished solution for injection of infusion.

These formulations are particularly preferably used when crobenetine is used as component 1.

D.2.1) Examples of pharmaceutical formulations of component 1:

### FORMULATION EXAMPLE 1

| | | |
|---|---|---|
| crobenetine hydrochloride | 767 | mg |
| HpγCD*) | 10000 | mg |
| mannitol | 11000 | mg |
| acetic acid 99% | 125.25 | mg |
| sodium acetate trihydrate | 56.5 | mg |
| water for injections ad | 250 | ml |

*)for example “CAVASOL ® W8 HP Pharma” made by Wacker

### FORMULATION EXAMPLE 2

| | | |
|---|---|---|
| crobenetine hydrochloride | 383.5 | mg |
| γCD | 5000 | mg |
| NaCl | 2250 | mg |
| water for injections ad | 250 | ml |

### FORMULATION EXAMPLE 3

| | | |
|---|---|---|
| crobenetine hydrochloride | 767 | mg |
| HPβCD | 7500 | mg |
| mannitol | 12500 | mg |
| acetic acid 99% | 125.25 | mg |
| sodium acetate trihydrate | 56.5 | mg |
| water for injections ad | 250 | ml |

### FORMULATION EXAMPLE 4

| | | |
|---|---|---|
| crobenetine hydrochloride | 767 | mg |
| SBEβCD | 5000 | mg |
| mannitol | 12500 | mg |
| acetic acid 99% | 125.25 | mg |
| sodium acetate trihydrate | 56.5 | mg |
| water for injections ad | 250 | ml |

### FORMULATION EXAMPLE 5

| | | |
|---|---|---|
| crobenetine hydrochloride | 767 | mg |
| HPγCD | 2500 | mg |
| citric acid | 708 | mg |
| mannitol | 12500 | mg |
| acetic acid 99% | 125.25 | mg |
| sodium acetate trihydrate | 56.5 | mg |
| water for injections ad | 250 | ml |

### FORMULATION EXAMPLE 6

| | | |
|---|---|---|
| crobenetine hydrochloride | 767 | mg |
| γCD | 2500 | mg |
| tartaric acid | 138.25 | mg |
| NaCl | 2250 | mg |
| water for injections ad | 250 | ml |

### FORMULATION EXAMPLE 7

### Solution for Infusion (Acetate Buffer pH 4)

| | | |
|---|---|---|
| crobenetine hydrochloride | 274 | mg |
| mannitol | 25000 | mg |
| acetic acid 99% | 250.5 | mg |
| sodium acetate trihydrate | 113.0 | mg |
| water for injections ad | 500 | ml |

### FORMULATION EXAMPLE 8

### Solution for Infusion (Acetate Buffer pH 4.5))

| | | |
|---|---|---|
| crobenetine hydrochloride | 274 | mg |
| mannitol | 25000 | mg |
| acetic acid 99% | 180.0 | mg |
| sodium acetate trihydrate | 265.0 | mg |
| water for injections ad | 500 | ml |

### FORMULATION EXAMPLE 9

### Solution for Infusion (Acetate Buffer pH 4))

| | | |
|---|---|---|
| crobenetine hydrochloride | 383.6 | mg |
| mannitol | 25000 | mg |
| acetic acid 99% | 501.0 | mg |
| sodium acetate trihydrate | 226.0 | mg |
| water for injections ad | 500 | ml |

### FORMULATION EXAMPLE 10

### Solution for Infusion (Acetate Buffer pH 4))

| | | |
|---|---|---|
| crobenetine hydrochloride | 767 | mg |
| mannitol | 11000 | mg |
| acetic acid 9% | 125.25 | mg |
| sodium acetate trihydrate | 56.5 | mg |
| water for injections ad | 250 | ml |

### FORMULATION EXAMPLE 11

### Solution for Infusion (Acetate Buffer PH 4.5))

| | | |
|---|---|---|
| crobenetine hydrochloride | 767 | mg |
| mannitol | 25000 | mg |
| acetic acid 99% | 90.0 | mg |
| sodium acetate trihydrate | 132.5 | mg |
| water for injections ad | 500 | ml |

### FORMULATION EXAMPLE 12

### Solution for Infusion (Acetate Buffer pH 4))

| | | |
|---|---|---|
| crobenetine hydrochloride | 219.1 | mg |
| mannitol | 5000 | mg |
| acetic acid 99% | 50.1 | mg |
| sodium acetate trihydrate | 22.6 | mg |
| water for injections ad | 100 | ml |

The amount of active substance given can be controlled by administering a particular volume of one of the solutions described above. For example the daily administration of 100 ml of a solution according to Example 1 corresponds to a dose of 280 mg of crobenetine a day.

D.3) Pharmaceutical formulation and administration of component 2:

The fibrinolytic 2 used within the scope of the drug combination according to the invention is generally a polypeptide which has to be given parenterally. It may be given particularly by intravenous, intraarterial, intramuscular, intra- or subcutaneous injection, but may also be administered by inhalation of a powder or aerosol. Typical formulations are freeze-dried preparations (lyophilisates) of the polypeptide, which are reconstituted immediately before administration with a solution for injection or infusion. The reconstituting solution may be water or a buffered aqueous solution. The formulation may, however, also consist of an aqueous solution which is preferably buffered with a physiologically acceptable buffer and may additionally contain conventional stabilisers, solubilisers and preservatives. Examples of conventional adjuvants for liquid or solid formulations of this kind are alkali metal hydrogen phosphate/alkali metal dihydrogen phosphate, sodium chloride, serum albumin, polyoxyethylenesorbitan-monolaurate (Tween® 20), polyoxyethylenesorbitan-monooleate (Tween® 80), ethylenediamine-tetraacetate (EDTA), sucrose, mannitol, dextran, amino acid and benzylalcohol (the latter only for liquid formulations). They are generally administered parenterally, preferably by intravenous injection or infusion. The mode of application and dosage preferably depend on the fibrinolytic selected, particularly its specific biological activity and half-life in the blood plasma. Thus, alteplase, which has a relatively short half-life, is typically administered in a total dosage of 100 mg as follows: 10-15 mg as an intravenous bolus, followed by an intravenous infusion of 50 mg over a period of 30 to 60 minutes, followed by another infusion of 60-180 minutes up to the maximum dose. Tenecteplase has a longer half-life and can therefore be administered as a single bolus, based on body weight, up to a maximum dose of 50 mg. Reteplase, which has a moderate half-life and low specific activity, is administered as an intravenous double bolus at an interval of 30 minutes in a dosage of 10 units (560 mg) per bolus. The skilled man knows how to determine the correct dosage for a new pharmaceutical composition. For the plasminogen activators mentioned by name, the skilled man can find information as to formulations and dosages in the literature mentioned above.

D.3.1) Pharmaceutical formulation examples of component 2:

Formulations of component 2 are known in the prior art and may be obtained commercially. Some commercially available formulations which may be used according to the invention are mentioned below by way of example and as an illustration.

Alteplase: (powder and solvent for preparing a solution for
injection/infusion)

| | |
|---|---|
| Composition: | Alteplase 10 mg/20 mg/50 mg/100 mg. |
| Other ingredients: | Arginine, phosphoric acid, Polysorbate 80. |
| | water for injections. |

Tenecteplase: (powder and solvent for preparing an injectable solution)

| | |
|---|---|
| Composition: | Tenecteplase 8 000 U/10 000 U (40 mg/50 mg). |
| Other ingredients: | Arginine, phosphoric acid, Polysorbate 20. |
| | water for injections 8 ml/10 ml. |

Reteplase: (powder and solvent for preparing an injectable solution)

| | |
|---|---|
| Composition: | Reteplase 0.56 g (corresponds to 10 units). |
| Other ingredients: | Tranexamic acid, potassium monohydrogen phosphate, |
| | phosphoric acid, sucrose, polysorbate 80. |
| | water for injections 10 ml. |

Streptokinase: (Dry substance for a solution for infusion)

| | |
|---|---|
| Composition: | highly-purified streptokinase 250000 I.U./750000 I.U./ |
| | 1500000 I.U. as dry substance. |
| Other ingredients: | Human albumin, sodium-L-hydrogen glutamate |
| | 1H₂O, Polygelin. |

Streptokinase: (oral tablets)

| | |
|---|---|
| Composition: | Streptokinase 10,000 I.U., Streptodornase 2500- |
| | 10,000 I.U. |
| Other ingredients: | Magnesium stearate, calcium hydrogen phosphate, |
| | maize starch, gum arabic. |

Urokinase: (dry substance)

| | |
|---|---|
| Composition: | Urokinase 500,000 I.U. |
| Other ingredients: | sodium monohydrogen phosphate, sodium |
| | dihydrogen phosphate, human albumin. |

Urokinase: (dry substance)

| | |
|---|---|
| Composition: | Urokinase (human) 500,000 I.U. |
| Other ingredients: | sodium dihydrogen phosphate, sodium |
| | monohydrogen phosphate, sodium chloride, |
| | Dextran 40. |

Anistreplase: (dry substance and solvent for i.v. injection)

| | |
|---|---|
| Composition: | 209-230 mg of dry substance with anistreplase |
| | 29.55-30.03 mg. |
| Other ingredients: | 4-amidinophenyl(p-anisate)-HCl 0.15-0.17 mg, |
| | dimethylsulphoxide 1-2 mg, aminocaproic acid 1.2- |
| | 1.6 mg, D-mannitol, human albumin, lysine-1HCl, |
| | sodium hydroxide, glycerol, water for injections. |

1) drug combinations containing one or more, preferably one sodium channel blocker 1 and one or more, preferably one fibrinolytic 2 optionally in the presence of conventional excipients or carriers.

2) Drug combinations according to claim 1, characterised in that 1 is selected from the group consisting of pirmencol, sipatrigine, irampanel, pilsicainide, oxcarbazepine, topiramate, fosphenytoin, flunarizin, ropivacaine, levobupivacaine, zonisamide, mexiletine, bipridil, bisaramil, milacainide, safinamide, bupivacaine, tetrodotoxin, NS 7, the compounds of general formula 1a 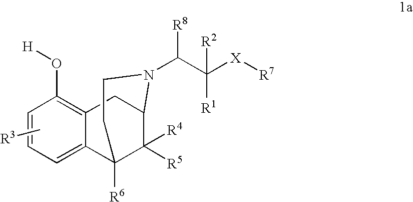
wherein
X denotes a single bond, —O, C₁-C₄-alkylene, an alkylene bridge with 1 to 8 carbon atoms which may be branched or unbranched and may have at any point in the bridge one or two oxygen atom(s) or a nitrogen atom, preferably O—C₁-C₃-alkylene or —O—CH₂—CH₂—O, —O—CH₂—CH₂—NH—;
R¹ denotes hydrogen, methyl, ethyl, phenyl;
R² denotes hydrogen, methyl;
R³ denotes hydrogen, fluorine, chlorine, bromine, hydroxy, methyl, methoxy;
R⁴ denotes hydrogen, methyl, ethyl;
R⁵ denotes hydrogen, methyl, ethyl;
R⁶ denotes hydrogen, methyl, ethyl;
R⁷ denotes tert.-butyl, cyclohexyl or phenyl, while phenyl may optionally be substituted by R⁹ and R¹⁰, which may be identical or different;
R⁸ denotes hydrogen, C₁-C₄-alkyl;
R⁹ denotes hydrogen, methyl, fluorine, chlorine, bromine, methoxy;
R¹⁰ denotes hydrogen, methyl, fluorine, chlorine, bromine, methoxy;
optionally in the form of the individual optical isomers, mixtures of the individual enantiomers or racemates as well as in the form of the free bases or the corresponding acid addition salts with pharmacologically acceptable acids;
and the compounds of general formula 1b 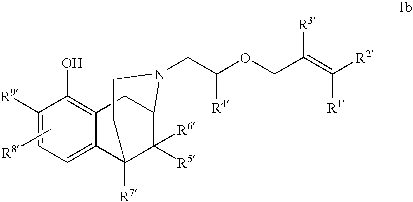
wherein
R^{1′}, R^{2′} and R^{3′} which may be identical or different, denote hydrogen, methyl or ethyl;
R^{4′} denotes hydrogen, methyl or ethyl;
R^{5′}, R^{6′} and R^{7′} which may be identical or different, denote hydrogen, methyl or ethyl;
R^{8′} and R^{9′} which may be identical or different, denote hydrogen, fluorine, chlorine, bromine, methyl, ethyl, hydroxy or methoxy,
optionally in the form of the racemates, the enantiomers, the diastereomers and the mixtures thereof, and optionally the pharmacologically acceptable acid addition salts thereof.

3) Drug combinations according to claim 2, characterised in that 1 is selected from the group consisting of pirmencol, pilsicainide, sipatrigine, irampanel, fosphenytoin, zonisamide, mexiletine, bipridil, bisaramil, milacainide, NS 7, the compounds of general formula 1a wherein
X denotes C₁-C₃-alkylene, —O—CH₂—CH₂—O or —O—CH₂—CH₂—NH—;
R¹ denotes hydrogen or methyl;
R² denotes hydrogen or methyl;
R³ denotes hydrogen or chlorine;
R⁴ denotes hydrogen or methyl;
R⁵ denotes hydrogen or methyl;
R⁶ denotes methyl or ethyl;
R⁷ denotes tert.-butyl, cyclohexyl or phenyl, while phenyl may optionally be substituted by R⁹ and R¹⁰, which may be identical or different;
R⁸ denotes hydrogen;
R⁹ denotes hydrogen, methyl, fluorine or chlorine;
R¹⁰ denotes hydrogen, methyl, fluorine or chlorine;
optionally in the form of the individual optical isomers, mixtures of the individual enantiomers or racemates as well as in the form of the free bases or the corresponding acid addition salts with pharmacologically acceptable acids;
and the compounds of general formula 1b, wherein
R^{1′}, R^{2′} and R^{3′} which may be identical or different, denote hydrogen or methyl;
R^{4′} denotes hydrogen or methyl;
R^{5′}, R^{6′} and R^{7′} which may be identical or different, denote hydrogen or methyl, preferably methyl;
R^{8′} denotes hydrogen, methyl, hydroxy or methoxy, preferably hydrogen or methyl,
R^{9′} denotes hydrogen or methyl,
optionally in the form of the racemates, the enantiomers, the diastereomers and the mixtures thereof, and optionally the pharmacologically acceptable acid addition salts thereof.

4) Drug combinations according to one of claims 1 to 3, characterised in that 2 is selected from among the plasminogen activators.

5) Drug combinations according to claim 4, characterised in that 2 is selected from the group consisting of alteplase (human tissue plasminogen activator, t-PA), tenecteplase, reteplase, streptokinase, urokinase, anistreplase, monteplase, nateplase, duteplase, lanoteplase, silteplase, amediplase and desmoteplase.

6) Drug combinations according to one of claims 1 to 5, characterised in that the active ingredients 1 and 2 are contained in a single, or in two separate, preferably in two separate preparations.

7) Use of a drug combination according to one of claims 1 to 6 for preparing a pharmaceutical composition for the treatment of ischaemic conditions of various origins.

8) Use according to claim 7, for preparing a pharmaceutical composition for the treatment of cardiac or cerebral ischaemias, most preferably for the treatment of stroke.

9) Use of one or more, preferably one sodium channel blocker 1 for preparing a pharmaceutical composition for the combined treatment of ischaemic conditions of various origins with one or more, preferably one fibrinolytic 2.

10) Use according to claim 9, characterised in that 1 is selected from among the compounds according to claim 2 and further characterised in that 2 is selected from among the compounds according to claim 4.
